(19) United States
(12) Patent Application Publication
(10) Pub. No.: US 2021/0285966 A1
(43) Pub. Date: Sep. 16, 2021

(54) DIAGNOSTIC METHODS FOR LIVER DISORDERS

(71) Applicant: Meso Scale Technologies, LLC., Rockville, MD (US)

(72) Inventor: George Sigal, Rockville, MD (US)

(21) Appl. No.: 17/213,954

(22) Filed: Mar. 26, 2021

Related U.S. Application Data
(63) Continuation of application No. 16/443,005, filed on Jun. 17, 2019, now abandoned.
Application No. 16/443,005 is a continuation of application No. 15/907,369, filed on Feb. 28, 2018, now abandoned.
Application No. 15/907,369 is a division of application No. 13/487,640, filed on Jun. 4, 2012, now abandoned.
(60) Provisional application No. 61/520,349, filed on Jun. 6, 2011.

(51) Int. Cl. G01N 33/68; G01N 33/576

(52) U.S. Cl. CPC G01N 33/6893; Y02A 50/30; G01N 2800/085; G01N 33/576

(57) ABSTRACT

The present invention relates to methods of diagnosing a liver disorder in a patient, as well as methods of monitoring the progression of a liver disorder and/or methods of monitoring a treatment protocol of a therapeutic agent or regimen. The invention also relates to assay kits used in connection with the diagnostic methods described herein.

### CROSS REFERENCE TO RELATED APPLICATION

The present application is a continuation of U.S. patent application Ser. No. 16/443,005, filed Jun. 17, 2019, which is a continuation of U.S. patent application Ser. No. 15/907,369, filed Feb. 28, 2018, now abandoned, which is a divisional of U.S. patent application Ser. No. 13/487,640, filed Jun. 4, 2012, now abandoned, which claims the benefit of U.S. Provisional Application No. 61/520,349, filed Jun. 6, 2011, the entire contents of which are incorporated herein by reference.

### FIELD OF THE INVENTION

This application relates to assay methods, modules and kits for conducting diagnostic assays useful in the detection and treatment of liver disorders.

### BACKGROUND OF THE INVENTION

Chronic liver disease is the eighth leading cause of death in the United States. Important causes of chronic liver disease include alcohol consumption, hepatitis, and to a lesser extent, congenital, metabolic, autoimmune, drug-induced conditions are also important contributors. Diagnosing liver disease can be a challenge, especially when patients are asymptomatic. In its early stages, liver disease may not be detected because the patient may remain asymptomatic until extensive liver damage occurs. Nonetheless, much of the morbidity and mortality associated with hepatic dysfunction can be prevented if liver disease is recognized before irreversible damage occurs. Diagnosing liver disease caused by viral hepatitis is especially urgent because of the risk of transmitting this infectious disease to others.

### SUMMARY OF THE INVENTION

The present invention provides a method and kits for monitoring liver health in a patient. The method includes the following steps:

(a) obtaining a test sample from a patient;

(b) measuring a level of a biomarker in said sample, wherein said biomarker is selected from bilirubin (total or fractionated, conjugated or unconjugated), ammonia, carbohydrate-deficient transferring (CDT), alanine aminotransferase (ALT), alkaline phosphatase (ALP), serum glutamic pyruvic transaminase (SGPT), aspartate aminotransferase (AST), serum glutamic oxaloacetic transaminase (SGOT), albumin, total protein (i.e., plasma proteins), gamma-glutamyl transferase (GGT), gamma-glutamyl transpeptidase (GGTP), lactic acid dehydrogenase (LDH), prothrombin time, or combinations thereof;

(c) measuring a level of a hepatitis biomarker selected from hepatitis A, B, C, D, E, or combinations thereof;

(d) comparing said level of said biomarker and said hepatitis biomarker in said sample to a level of said biomarker and said hepatitis biomarker in a normal control sample; and

(e) diagnosing the presence or absence of a liver disorder in said patient based on said comparison.

The invention also contemplates a multiplexed assay kit used to monitor liver health in a patient sample, said kit comprising an assay chamber configured to conduct a multiplexed assay measurement for:

(a) a level of a biomarker in said sample comprising bilirubin (total or fractionated, conjugated or unconjugated), ammonia, carbohydrate-deficient transferring (CDT), alanine aminotransferase (ALT), alkaline phosphatase (ALP), serum glutamic pyruvic transaminase (SGPT), aspartate aminotransferase (AST), serum glutamic oxaloacetic transaminase (SGOT), albumin, total protein (i.e., plasma proteins), gamma-glutamyl transferase (GGT), gamma-glutamyl transpeptidase (GGTP), lactic acid dehydrogenase (LDH), prothrombin time, or combinations thereof; and

(b) a level of a hepatitis biomarker in said sample, wherein said hepatitis biomarker is specific for a form of hepatitis selected from hepatitis A, B, C, D, or E.

In one embodiment, the assay chamber is a single well of an assay plate. Alternatively, the assay chamber is a cartridge. The assay chamber is configured to conduct an immunoassay-based multiplexed assay measurement. The kit can include one or more additional assay reagents used in said multiplexed assay measurement, said one or more additional assay reagents provided in one or more vials, containers, or compartments of said kit. For example, the kit can include a multi-well assay test plate and said one or more additional assay reagents are provided in a compartment of said multi-well assay test plate. In this embodiment, the test plate comprises a plurality of assay domains, at least two of said assay domains comprising reagents for measuring (a) different biomarkers, (b) different hepatitis biomarkers, (c) said biomarker and said hepatitis biomarker, and (d) combinations thereof. For example, a first assay domain in said well comprises a reagent to measure said biomarker and a second assay domain in said well comprises an additional reagent to measure said hepatitis biomarker.

The methods and kits of the invention can be configured in a variety of ways, incorporating numerous reagents, without departing from the spirit or scope of the invention. For example, the invention contemplates a method and accompanying kit for monitoring liver health in a patient comprising: (a) ordering a test comprising a measurement of the levels of one or more biomarkers and one or more hepatitis biomarkers in a test sample obtained from a patient; (b) comparing the levels identified in step (a) to a normal control; and (c) evaluating from said comparing step (b) the relative liver health of said patient. The biomarkers identified by the applicants can be used to diagnose one or more of liver disorders in a patient, to assess the progression of one or more of liver disorders in a patient, or to assess the efficacy of a treatment regimen for one or more liver disorders. For example, a patient that has been previously diagnosed with hepatitis is evaluated for progression of that disorder to liver disease (e.g., cirrhosis, fibrosis, alcoholic liver disease, fatty liver disease, and combinations thereof). The level(s) of the various biomarkers identified herein may reflect the responsiveness or non-responsiveness of a liver disease condition to a given treatment regimen. A response to a treatment regimen for liver disease includes a detectable reduction of one or more of the symptoms of liver disease, including but not limited to improved liver function, as evidenced by levels of liver enzymes and byproducts.

In one embodiment, the methods and kits of the invention include measuring a hepatitis A marker (e.g., an antibody to hepatitis A virus, e.g., an IgM antibody), a hepatitis B biomarker (e.g., a hepatitis B surface antigen, an antibody for said hepatitis B surface antigen, a hepatitis B core antigen, an antibody for said hepatitis B core antigen, a hepatitis B e antigen, an antibody to said hepatitis B e antigen, and combinations thereof, wherein one or more of the antibodies may be IgM antibodies), a hepatitis C biomarker (e.g., hepatitis C surface antigen, an antibody to said hepatitis C surface antigen, and combinations thereof, wherein the antibody may be an IgM antibody), a hepatitis D biomarker, a hepatitis E biomarker, and combinations thereof. In a particularly preferred embodiment, the method and kit measures a hepatitis biomarker selected from a hepatitis B biomarker and a hepatitis C biomarker.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The invention provides a method of monitoring liver health in a patient by measuring a variety of biomarkers associated with liver health that may be indicative of one or more disease conditions of the liver. In particular, the method includes measuring (a) a biomarker or panel of liver health biomarkers including one or more of the following: bilirubin (total or fractionated, conjugated or unconjugated), ammonia, carbohydrate-deficient transferring (CDT), alanine aminotransferase (ALT), alkaline phosphatase (ALP), serum glutamic pyruvic transaminase (SGPT), aspartate aminotransferase (AST), serum glutamic oxaloacetic transaminase (SGOT), albumin, total protein (i.e., plasma proteins), gamma-glutamyl transferase (GGT), gamma-glutamyl transpeptidase (GGTP), lactic acid dehydrogenase (LDH), iron status, prothrombin time, and combinations thereof; and (b) a level of a hepatitis biomarker selected from the group consisting of a hepatitis A biomarker, a hepatitis B biomarker, a hepatitis C biomarker, a hepatitis D biomarker, a hepatitis E biomarker, and combinations thereof; and comparing the levels of the liver health biomarker and the hepatitis biomarker in the sample to a level of the liver health biomarker and the hepatitis biomarker in a normal control sample and diagnosing the presence or absence of a liver disorder in the patient based on the results of this comparison.

In a preferred embodiment, the hepatitis biomarker is a hepatitis A, B, and/or C biomarker, and in a particularly preferred embodiment, the hepatitis biomarker is a hepatitis B and/or C biomarker. In one embodiment, the method includes measuring hepatitis B and C, as well as a biomarker or panel of biomarkers selected from bilirubin (total or fractionated, conjugated or unconjugated), ammonia, carbohydrate-deficient transferring (CDT), alanine aminotransferase (ALT), alkaline phosphatase (ALP), serum glutamic pyruvic transaminase (SGPT), aspartate aminotransferase (AST), serum glutamic oxaloacetic transaminase (SGOT), albumin, total protein (i.e., plasma proteins), gamma-glutamyl transferase (GGT), gamma-glutamyl transpeptidase (GGTP), lactic acid dehydrogenase (LDH), iron status, prothrombin time, and combinations thereof.

Any suitable biomarker of hepatitis A, B, C, D, or E may be analyzed in the instant method. In one embodiment the biomarker comprises a surface antigen of hepatitis A, B, C, D, or E, or an antibody to that surface antigen, e.g., an IgM antibody. If the hepatitis biomarker is hepatitis A, a preferred biomarker is an antibody to hepatitis A, and preferably an IgM antibody. If the hepatitis biomarker is hepatitis B, the biomarker may be selected from a hepatitis B surface antigen, an antibody for the hepatitis B surface antigen, a hepatitis B core antigen, an antibody for the hepatitis B core antigen, a hepatitis B e antigen, an antibody to the hepatitis B e antigen, and combinations thereof. One or more of the antibodies to hepatitis B antigens may be IgM antibodies. If the hepatitis biomarker is a hepatitis C biomarker, the biomarker may be selected from hepatitis C surface antigen, an antibody to the hepatitis C surface antigen, and combinations thereof, and in one embodiment, the antibody to the hepatitis C surface antigen is an IgM antibody. In the instant method, the level of the hepatitis biomarker is measured in the sample and compared to that of a normal control. In a preferred embodiment, the measurement of a detectable amount of a hepatitis biomarker is indicative of a disease condition as a normal control would not include a detectable amount of a hepatitis biomarker.

Approximate levels for a normal control sample of the liver health biomarkers listed above are provided in Table 1 (all levels are for an adult male or female). These levels are non-limiting and may vary by laboratory and/or patient. It is within the skill of the ordinary artisan to identify elevated levels of one or more of the biomarkers listed in Table 1 in a patient sample.

TABLE 1
Approximate Levels in Normal Control Samples

| Liver Health Biomarker | Approximate Normal Range |
|---|---|
| Bilirubin (total) | 0.3 to 1.9 mg/dL |
| Bilirubin (conjugated) | 0 to 0.3 mg/dL |
| ammonia | 0 to 40 micromol/L |
| carbohydrate-deficient | 20 to 26 U/l (units/liter) |
| transferring (CDT) | |
| alanine aminotransferase (ALT) | 167 to 667 nkat/L (10 to 40 U/L) |
| alkaline phosphatase (ALP) | 25 to 100 units per liter (U/L) or |
| | 0.43-1.70 microkat/liter (mckat/L) |
| serum glutamic pyruvic | 5 to 60 U/L |
| transaminase (SGPT) | |
| aspartate aminotransferase (AST) | 8 to 35 units per liter (U/L) or |
| | 5 to 40 International Units per |
| | liter (IU/L) |
| serum glutamic oxaloacetic | 6 to 40 IU/L |
| transaminase (SGOT) | |
| albumin | 3.4 to 5.4 g/dL |
| total protein (i.e., plasma | 6.0 to 8.3 gm/dL |
| proteins) | |
| gamma-glutamyl transferase (GGT) | 0 to 51 IU/L |
| lactic acid dehydrogenase (LDH) | 105-333 IU/L |
| iron status | 50 to 175 ug/dL |
| prothrombin time | 11 to 13.5 seconds (for patients |
| | not taking a blood thinner |
| | medication) |

In addition to the liver health biomarkers listed above, other biomarkers of disease may also be analyzed, e.g., biomarkers of hepatocellular carcinoma (HCC), including but not limited to CEA, CA 125, CA 19-9, OPN, MMP-9, E-cadherin, and erbB2, as well as glypican 3, PIVKA II, ER6Q, Vimentin, actin alpha 1 skeletal muscle protein, hMFAP 4, tropomyosin, PTGES 2, amyloid P component, transgelin, calponin 1, Homo sapiens p20 protein, 17 kDa myosin light chain, H chain H Igg B12, prolyl 4-hydroxylase, beta subunit methylenetetrahydrofolate dehydrogenase 1, PRO2619, aldehyde dehydrogenase 1, fibrinogen alpha chain preproprotein, fructose-bisphosphate aldolase B, argininosuccinate synthetase, Eefla2, AT P 5 Al, alpha-2 actin, regucalcin, mitochondrial malate dehydrogenase, mitochondrial acetoacetyl-CoA thiolase, Prothrombin, Gamma Glutamyl Transpeptidase, Apolipoprotein Al (PGA) index, Age platelet (AP) index, Bonacini index, Pohl score, Foms index, Aspartate aminotransferase/Platelets Ratio index (APRI), MP3 (MMP1, PIINP) index, FIB4, Fibrolndex, and combinations thereof. In one embodiment, the combination of hepatitis biomarkers, liver disease biomarkers and liver cancer biomarkers allows for better differentiation of liver cancer from non-cancerous liver diseases.

In one embodiment of the present invention, the biomarker level(s) in the test sample, i.e., the levels of the liver health biomarker(s) and the hepatitis biomarker(s) is/are compared to the biomarker level(s) in a corresponding normal control sample. The difference between the normal control sample biomarker levels and that of the test sample may be the basis for diagnosing a liver disorder in a patient. Alternatively, the biomarker level(s) may be compared to a detection cut-off level or range, wherein the biomarker level above or below the detection cut-off level (or within the detection cut-off range) is indicative of the liver disorder. For example, i) having a level of at least one of the biomarkers above or below a detection cut-off level (or within a detection cut-off range) for that biomarker is indicative of a liver disorder; ii) having the level of two or more (or all) of the biomarkers above or below a detection cut-off level (or within a detection cut-off range) for each of the biomarkers is indicative of the liver disorder, or iii) an algorithm based on the levels of the multiple biomarkers is used to determine if the liver disorder is present.

In addition, the methods of the present invention may be used in combination with other methods of diagnosing liver disease in a patient. In one embodiment, the patient may also be subjected to one or more diagnostic tools designed to detect liver disease. For example, imaging methods may be used to provide images of the liver to look for tumors and blocked bile ducts and can be used to evaluate liver size and blood flow through the liver. In addition, a liver biopsy may be performed. Imaging methods that may be performed include abdominal ultrasound, computed tomography (CT) scan of the abdomen (including the liver, gallbladder, and spleen), magnetic resonance imaging (MRI) scan of the abdomen, and a liver and spleen scan. Still further, other tests that may be performed include, paracentesis, endoscopy, endoscopic retrograde cholangiopancreatogram, and ammonia testing.

The biomarkers identified by the applicants may be used to diagnose one or more of liver disorders in a patient, to assess the progression of one or more of liver disorders in a patient, or to assess the efficacy of a treatment regimen for one or more liver disorders. In one embodiment of the invention, a patient that has been previously diagnosed with hepatitis is evaluated for progression of that disorder to liver disease (e.g., cirrhosis, fibrosis, alcoholic liver disease, fatty liver disease, and combinations thereof). The level(s) of the various biomarkers identified herein may reflect the responsiveness or non-responsiveness of a liver disease condition to a given treatment regimen. A response to a treatment regimen for liver disease includes a detectable reduction to some extent of one or more of the symptoms of liver disease, including but not limited to improved liver function, as evidenced by levels of liver enzymes and byproducts. A response to a chemotherapeutic therapeutic regimen includes a detectable reduction to some extent of one or more of the symptoms of a cancerous disorder, including, but not limited to: (1) reduction in the number of cancer cells: (2) reduction in tumor size; (3) inhibition (i.e., slowing to some extent, preferably stopping) of cancer cell infiltration into peripheral organs; (4) inhibition (i.e., slowing to some extent, preferably stopping) of tumor metastasis; (5) inhibition, to some extent, of tumor growth; (6) relieving or reducing to some extent one or more of the symptoms associated with the disorder; and/or (7) increasing, to some extent, the overall survival of a patient relative to that observed for the standard of care for hepatocellular carcinoma. A response to a therapeutic regimen may also comprise maintenance of a therapeutic benefit, including, but not limited to (1) inhibiting an increase in the number of cancer cells: (2) inhibiting an increase in tumor size; (3) inhibiting cancer cell infiltration into peripheral organs; (4) inhibiting tumor metastases; (5) relieving or reducing to some extent one or more of the symptoms associated with the disorder; and/or (6) inhibiting a recurrence or onset of one or more of the symptoms associated with the disorder.

In addition, the level of a biomarker may be determined at any time point before and/or after initiation of treatment. In one embodiment, the biomarker is used to gauge the efficacy of a therapeutic regimen. Therefore, the method of the present invention may include measuring a baseline level(s) of a biomarker before a therapeutic regimen is initiated, and the method may further comprise comparing the level and the baseline level. Moreover, the method may further comprise measuring an interim level of the biomarker during a therapeutic regimen and the method further comprises comparing the level, the interim level and the baseline level.

Alternatively, the measuring step may comprise measuring a level(s) of a biomarker before a therapeutic regimen is initiated to predict whether a liver disorder will be responsive or non-responsive to a given therapeutic regimen. The method may further comprise modifying the therapeutic regimen based on the level(s) of a biomarker observed during the measuring step, e.g., increasing or decreasing the dosage, frequency, or route of administration of a therapeutic agent, adding an additional therapeutic agent and/or palliative agent to a treatment regimen, or if the therapeutic regimen includes the administration of two or more therapeutic and/or palliative agents, the treatment regimen may be modified to eliminate one or more of the therapeutic and/or palliative agents used in the combination therapy.

As described herein, the measured levels of one or more biomarkers may be used to detect or monitor a liver disorder and/or to determine the responsiveness of a liver disorder to a specific treatment regimen. The specific methods/algorithms for using biomarker levels to make these determinations, as described herein, may optionally be implemented by software running on a computer that accepts the biomarker levels as input and returns a report with the determinations to the user. This software may run on a standalone computer or it may be integrated into the software/computing system of the analytical device used to measure the biomarker levels or, alternatively, into a laboratory information management system (LIMS) into which crude or processed analytical data is entered. In one embodiment, biomarkers are measured in a point-of-care clinical device which carries out the appropriate methods/algorithms for detecting, monitoring or determining the responsiveness of a cancer and which reports such determination(s) back to the user.

The assays of the present invention may be conducted by any suitable method. In one embodiment, the measuring step is conducted on a single sample, and it may be conducted in a single assay chamber or assay device, including but not limited to a single well of an assay plate, a single assay cartridge, a single lateral flow device, a single assay tube, etc.

As used herein, the term “sample” is intended to mean any biological fluid, cell, tissue, organ or combinations or portions thereof, which includes or potentially includes a biomarker of a disease of interest. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. In one embodiment, the samples that are analyzed in the assays of the present invention are blood or blood fractions such as, serum and plasma. Other suitable samples include biopsy tissue, intestinal mucosa and urine. In one embodiment, the level is measured using an immunoassay.

As used herein, a “biomarker” is a substance that is associated with a particular disease. A change in the levels of a biomarker may correlate with the risk or progression of a disease or with the susceptibility of the disease to a given treatment. A biomarker may be useful in the diagnosis of disease risk or the presence of disease in an individual, or to tailor treatments for the disease in an individual (choices of drug treatment or administration regimes). In evaluating potential drug therapies, a biomarker may be used as a surrogate for a natural endpoint such as survival or irreversible morbidity. If a treatment alters a biomarker that has a direct connection to improved health, the biomarker serves as a “surrogate endpoint” for evaluating clinical benefit A sample that is assayed in the diagnostic methods of the present invention may be obtained from any suitable patient, including but not limited to a patient suspected of having cancer, cirrhosis, HBV, HCV or alcoholic liver disease or a patient having a predisposition to one or more of these conditions. The patient may or may not exhibit symptoms associated with one or more of these conditions.

As used herein, the term “level” refers to the amount, concentration, or activity of a biomarker. The term “level” may also refer to the rate of change of the amount, concentration or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide, nucleic acid or small molecule. The term can be used to refer to an absolute amount of a biomarker in a sample or to a relative amount of the biomarker, including amount or concentration determined under steady-state or non-steady-state conditions. Level may also refer to an assay signal that correlates with the amount, concentration, activity or rate of change of a biomarker. The level of a biomarker can be determined relative to a control biomarker in a sample.

According to one aspect of the invention, the level(s) of biomarker(s) are measured in samples collected from individuals clinically diagnosed with, suspected of having or at risk of developing a liver disorder. Initial diagnosis may have been carried out using conventional methods, e.g., biopsy or other conventional diagnostic methods. The level(s) of biomarker(s) are also measured in healthy individuals. Specific biomarkers valuable in distinguishing between normal and diseased patients are identified by visual inspection of the data, for example, by visual classification of data plotted on a one-dimensional or multidimensional graph, or by using statistical methods such as characterizing the statistically weighted difference between control individuals and diseased patients and/or by using Receiver Operating Characteristic (ROC) curve analysis. A variety of suitable methods for identifying useful biomarkers and setting detection thresholds/algorithms are known in the art and will be apparent to the skilled artisan.

For example and without limitation, diagnostically valuable biomarkers may be first identified using a statistically weighted difference between control individuals and diseased patients, calculated as

$$\frac{D - N}{\sqrt{{σ}_{D} * {σ}_{N}}}$$

wherein D is the median level of a biomarker in patients diagnosed as having, for example, liver cancer, N is the median (or average) of the control individuals, σ_{D} is the standard deviation of D and σ_{N} is the standard deviation of N. The larger the magnitude, the greater the statistical difference between the diseased and normal populations.

According to one embodiment of the invention, biomarkers resulting in a statistically weighted difference between control individuals and diseased patients of greater than, e.g., 1, 1.5, 2, 2.5 or 3 could be identified as diagnostically valuable biomarkers.

Another method of statistical analysis for identifying biomarkers is the use of z-scores, e.g., as described in Skates et al. (2007) Cancer Epidemiol. Biomarkers Prev. 16(2):334-341.

Another method of statistical analysis that can be useful in the inventive methods of the invention for determining the efficacy of particular candidate analytes, such as particular biomarkers, for acting as diagnostic marker(s) is ROC curve analysis. An ROC curve is a graphical approach to looking at the effect of a cut-off criterion, e.g., a cut-off value for a diagnostic indicator such as an assay signal or the level of an analyte in a sample, on the ability of a diagnostic to correctly identify positive or negative samples or subjects. One axis of the ROC curve is the true positive rate (TPR, i.e., the probability that a true positive sample/subject will be correctly identified as positive, or alternatively, the false negative rate (FNR=1-TPR, the probability that a true positive sample/subject will be incorrectly identified as a negative). The other axis is the true negative rate, i.e., TNR, the probability that a true negative sample will be correctly identified as a negative, or alternatively, the false positive rate (FPR=1-TNR, the probability that a true negative sample will be incorrectly identified as positive). The ROC curve is generated using assay results for a population of samples/subjects by varying the diagnostic cut-off value used to identify samples/subjects as positive or negative and plotting calculated values of TPR or FNR and TNR or FPR for each cut-off value. The area under the ROC curve (referred to herein as the AUC) is one indication of the ability of the diagnostic to separate positive and negative samples/subjects. In one embodiment, a biomarker provides an AUC≥0.7. In another embodiment, a biomarker provides an AUC≥0.8. In another embodiment, a biomarker provides an AUC≥0.9.

Diagnostic indicators analyzed by ROC curve analysis may be a level of an analyte, e.g., a biomarker, or an assay signal. Alternatively, the diagnostic indicator may be a function of multiple measured values, for example, a function of the level/assay signal of a plurality of analytes, e.g., a plurality of biomarkers, or a function that combines the level or assay signal of one or more analytes with a patient's scoring value that is determined based on visual, radiological and/or histological evaluation of a patient. The multi-parameter analysis may provide more accurate diagnosis relative to analysis of a single biomarker.

Candidates for a multi-analyte panel could be selected by using criteria such as individual analyte ROC areas, median difference between groups normalized by geometric interquartile range (IQR) etc. The objective is to partition the analyte space to improve separation between groups (for example, normal and disease populations) or to minimize the misclassification rate.

One approach is to define a panel response as a weighted combination of individual analytes and then compute an objective function like ROC area, product of sensitivity and specificity, etc. See e.g., WO 2004/058055, as well as US2006/0205012, the disclosures of which are incorporated herein by reference in their entireties.

Biomarker levels may be measured using any of a number of techniques available to the person of ordinary skill in the art, e.g., direct physical measurements (e.g., mass spectrometry) or binding assays (e.g., immunoassays, agglutination assays and immunochromatographic assays). The method may also comprise measuring a signal that results from a chemical reactions, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

Binding assays for measuring biomarker levels may use solid phase or homogenous formats. Suitable assay methods include sandwich or competitive binding assays. Examples of sandwich immunoassays are described in U.S. Pat. Nos. 4,168,146 and 4,366,241, both of which are incorporated herein by reference in their entireties. Examples of competitive immunoassays include those disclosed in U.S. Pat. Nos. 4,235,601, 4,442,204 and 5,208,535, each of which are incorporated herein by reference in their entireties.

Multiple biomarkers may be measured using a multiplexed assay format, e.g., multiplexing through the use of binding reagent arrays, multiplexing using spectral discrimination of labels, multiplexing of flow cytometric analysis of binding assays carried out on particles, e.g., using the Luminex® system. Suitable multiplexing methods include array based binding assays using patterned arrays of immobilized antibodies directed against the biomarkers of interest. Various approaches for conducting multiplexed assays have been described (See e.g., US 20040022677; US 20050052646; US 20030207290; US 20030113713; US 20050142033; and US 20040189311, each of which is incorporated herein by reference in their entireties. One approach to multiplexing binding assays involves the use of patterned arrays of binding reagents, e.g., U.S. Pat. Nos. 5,807,522 and 6,110,426; Delehanty J-B., Printing functional protein microarrays using piezoelectric capillaries, Methods Mol. Bio. (2004) 278: 135-44; Lue R Y et al., Site-specific immobilization of biotinylated proteins for protein microarray analysis, Methods Mol. Biol. (2004) 278: 85-100; Lovett, Toxicogenomics: Toxicologists Brace for Genomics Revolution, Science (2000) 289: 536-537; Bems A, Cancer: Gene expression in diagnosis, nature (2000), 403, 491-92; Walt, Molecular Biology: Bead-based Fiber-Optic Arrays, Science (2000) 287: 451-52 for more details). Another approach involves the use of binding reagents coated on beads that can be individually identified and interrogated. See e.g., WO 9926067, which describes the use of magnetic particles that vary in size to assay multiple analytes; particles belonging to different distinct size ranges are used to assay different analytes. The particles are designed to be distinguished and individually interrogated by flow cytometry. Vignali has described a multiplex binding assay in which 64 different bead sets of microparticles are employed, each having a uniform and distinct proportion of two dyes (Vignali, D. A A, “Multiplexed Particle-Based Flow Cytometric Assays” J. ImmunoL Meth. (2000) 243: 243-55). A similar approach involving a set of 15 different beads of differing size and fluorescence has been disclosed as useful for simultaneous typing of multiple pneumococcal serotypes (Park, M. K et al., “A Latex Bead-Based Flow Cytometric Immunoassay Capable of Simultaneous Typing of Multiple Pneumococcal Serotypes (Multibead Assay)” Clin. Diag. Lab ImmunoL (2000) 7: 4869). Bishop, J E et al. have described a multiplex sandwich assay for simultaneous quantification of six human cytokines (Bishop, L E. et al., “Simultaneous Quantification of Six Human Cytokines in a Single Sample Using Microparticle-based Flow Cytometric Technology,” Clin. Chem (1999) 45:1693-1694).

A diagnostic test may be conducted in a single assay chamber, such as a single well of an assay plate or an assay chamber that is an assay chamber of a cartridge. The assay modules, e.g., assay plates or cartridges or multi-well assay plates), methods and apparatuses for conducting assay measurements suitable for the present invention are described for example, in US 20040022677; US 20050052646; US 20050142033; US 20040189311, each of which is incorporated herein by reference in their entireties. Assay plates and plate readers are now commercially available (MULTI-SPOT® and MULTI-ARRAY® plates and SECTOR® instruments, Meso Scale Discovery®, a division of Meso Scale Diagnostics, LLC, Gaithersburg, Md.).

Various publications and test methods are cited herein, the disclosures of which are incorporated herein by reference in their entireties, In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

1. A method for monitoring liver health in a patient, said method comprising
(a) obtaining a test sample from a patient, wherein said sample is collected at a first time point;
(b) measuring a level of a biomarker in said sample, wherein said biomarker is selected from bilirubin (total or fractionated, conjugated or unconjugated), ammonia, carbohydrate-deficient transferring (CDT), alanine aminotransferase (ALT), alkaline phosphatase (ALP), serum glutamic pyruvic transaminase (SGPT), aspartate aminotransferase (AST), serum glutamic oxaloacetic transaminase (SGOT), albumin, total plasma proteins, gamma-glutamyl transferase (GGT), gamma-glutamyl transpeptidase (GGTP), lactic acid dehydrogenase (LDH), prothrombin time, or combinations thereof;
(c) measuring a level of a biomarker of hepatocellular carcinoma (HCC) in said sample, the biomarker of HCC selected from the group consisting of CEA, CA 125, CA 19-9, OPN, MMP-9, E-cadherin, and erbB2, and said method further comprises measuring said levels of said biomarker and said HCC biomarker in an additional sample, wherein said additional sample is collected from said patient as a second time point;
(d) comparing said level of said biomarker and said HCC biomarker at said first and second time points to a level of said biomarker and said HCC biomarker in a normal control sample; and
(e) diagnosing the presence or absence of HCC in said patient based on said comparison, wherein the presence or absence of HCC is when the statistically weighted difference of the plurality of biomarkers between HCC is 1 or greater.

2. The method of claim 1 further comprising measuring a hepatitis biomarker selected from a hepatitis A marker, a hepatitis B biomarker, a hepatitis C biomarker, a hepatitis D biomarker, and a hepatitis E biomarker.

3. The method of claim 2 wherein said hepatitis biomarker comprises a hepatitis A biomarker including an antibody to hepatitis A virus.

4. The method of claim 3 wherein said antibody is an IgM antibody.

5. The method of claim 2 wherein said hepatitis biomarker comprises a hepatitis B biomarker selected from a hepatitis B surface antigen, an antibody for said hepatitis B surface antigen, a hepatitis B core antigen, an antibody for said hepatitis B core antigen, a hepatitis B e antigen, an antibody to said hepatitis B e antigen, or combinations thereof.

6. The method of claim 2 wherein said hepatitis biomarker comprises a hepatitis C biomarker selected from a hepatitis C surface antigen, an antibody to said hepatitis C surface antigen, or combinations thereof.

7. The method of claim 2 wherein said hepatitis biomarker comprises a hepatitis B biomarker and a hepatitis C biomarker.

8. The method of claim 7 wherein said hepatitis B biomarker is selected from a hepatitis B surface antigen, an antibody for said hepatitis B surface antigen, a hepatitis B core antigen, an antibody for said hepatitis B core antigen, a hepatitis B e antigen, an antibody to said hepatitis B e antigen, or combinations thereof

9. The method of claim 7 wherein said hepatitis C biomarker is selected from a hepatitis C surface antigen, an antibody to said hepatitis C surface antigen, or combinations thereof.

10. The method of claim 5 wherein said hepatitis B biomarker is selected from said hepatitis B surface antigen, said antibody to said hepatitis B surface antigen, said antibody to said hepatitis B core antigen, or combinations thereof.

11. The method of claim 2, wherein said comparing step comprises comparing said level of said biomarker and said hepatitis biomarker to a detection cut-off level for each of said biomarker and said hepatitis biomarker.

12. The method of claim 1, wherein said patient has been diagnosed with liver disease.

13. The method of claim 12, wherein said liver disorder is selected from cirrhosis, fibrosis, hepatitis, alcoholic liver disease, fatty liver disease, or combinations thereof.

14. The method of claim 2, wherein said levels of said biomarker and said hepatitis biomarker are measured in a multiplexed assay.

15. The method of claim 2, wherein said levels of said biomarker and said hepatitis biomarker are each measured in a single assay chamber.

16. The method of claim 15, wherein said assay chamber is a single well of an assay plate.

17. The method of claim 15, wherein said assay chamber is an assay chamber of a cartridge.

18. The method of claim 1, wherein said sample is selected from blood, serum or plasma.

19. The method of claim 1, wherein said sample is selected from biopsy tissue, intestinal mucosa or urine.

20. The method of claim 14, wherein said level is measured using an immunoassay.
